(19) United States
(12) Patent Application Publication
(10) Pub. No.: US 2007/0032720 A1
(43) Pub. Date: Feb. 8, 2007

(54) Method and system for navigating in real time in three-dimensional medical image model

(75) Inventors: John Koivukangas, Oulu (FI); Vesa Pentikainen, Kiiminki (FI)

Correspondence Address: YOUNG & THOMPSON, 745 SOUTH 23RD STREET, 2ND FLOOR, ARLINGTON, VA, 22202, US

(73) Assignee: ONESYS OY, Oulu (FI)

(21) Appl. No.: 10/561,241

(22) PCT Filed: Jun. 16, 2004
(86) PCT No.: PCT/FI04/00371

§ 371 Date: Dec. 19, 2005

(30) Foreign Application Priority Data
Jun. 17, 2003 (FI) 20030910

(51) Int. Cl. A61B 5/05

(52) U.S. Cl. 600/407

(57) ABSTRACT

The invention relates to a system for navigating in real time in a three-dimensional medical image model and to a method thereof. The method includes displaying a orientation view of the medical image model on a display; adjusting a location related to the displayed orientation view of the medical image model based on a pointing device alignment; displaying an inside view related to the location into the medical image model; and adjusting a viewing direction to the inside view of the medical image model based on a detected orientation of the pointing device.

### FIELD

The invention relates to a system and a method for navigating in real time in a medical image model within a three-dimensional virtual work-space.

### BACKGROUND

Medical diagnosis and surgical planning typically comprise studying two-dimensional images of a patient on an illuminated light box or a computer display, for example. The two-dimensional images are, for example, MRI (magnetic resonance imaging)—slices of a target area of the patient. MRI is used to visualize some procedures such as brain surgery. In order to make the diagnosis or planning treatments the two-dimensional image slices are routinely studied. However, the understanding of the target area of the patient based on the two-dimensional image slices is time-consuming and a difficult process. One reason for that is that the visualization is a two-dimensional process while the actual surgical procedure is three-dimensional.

Minimally invasive treatment of the human body is becoming popular. The treatment can be planned by virtual reality visualization of the treatment area. Known minimally invasive surgical procedures are often visually guided; but such methods often do not permit visualization within the target tissue or organ. Intuitive real-time three-dimensional visualization of the tissues would provide accurate guidance of therapy.

Systems for representing information as rendered three-dimensional images have proved to be suited to representing large amounts of information and/or complex information in an efficient and in a compact manner. Users can often more easily understand the displays produced within such information visualization systems than other conventional representations. The user viewpoint within the visualization systems is the viewpoint to which the three-dimensional representation of the three-dimensional image model is rendered. The rendering system responds to input from the user to change the desired viewpoint accordingly. When a new viewpoint position and/or distance are input by the user, the rendering system re-renders the view appropriately. When real time or near-real time rendering is provided, the user is able to up-date the viewpoint and study the result immediately.

However, the known visualization systems for medical image models are uncomfortable for the users. The user is not able to navigate through the three-dimensional medical image model with simple motions of the input device. There is a need for user-friendlier real time navigating systems in three-dimensional medical image models.

### BRIEF DESCRIPTION OF THE INVENTION

An object of the invention is to provide an improved method and a system for navigating in real time in a three-dimensional medical image model. According to an aspect of the invention, there is provided a method for navigating in real time in a three dimensional medical image model the method comprising: displaying an orientation view of the medical image model on a display; adjusting a location related to the displayed orientation view of the medical image model based on a pointing device alignment; displaying an inside view related to the location into the medical image model; and adjusting a viewing direction to the inside view of the medical image model based on a detected orientation of the pointing device.

According to another aspect of the invention, there is provided a system for navigating in real time in a three-dimensional medical image model, the system comprising a control unit for controlling the functions of the system, a pointing device connected to the control unit and a display connected to the control unit, the control unit being configured to: display an orientation view of the medical image model on the display; adjust a location related to the displayed orientation view of the medical image model based on the pointing device alignment; display an inside view related to the location into the medical image model; and adjust a viewing direction to the inside view of the medical image model based on a detected orientation of the pointing device.

Preferred embodiments of the invention are described in the dependent claims.

The method and system of the invention provide several advantages. The viewing of the three-dimensional medical image models becomes simple. A large amount of information can be viewed very efficiently. Navigating the details of a three-dimensional medical image model is possible in a user-friendly manner and even when the patient is not present.

### LIST OF DRAWINGS

In the following, the invention will be described in greater detail with reference to the embodiments and the accompanying drawings, in which

FIG. 1 shows an example of the structure of a system for navigating in a three-dimensional medical image model;

FIG. 2 shows another example of the structure of a system for navigating in a three-dimensional medical image model,

FIG. 3 shows an example of the method for navigating in a three-dimensional medical image model,

FIGS. 4A and 4B show an example of the implementation of the method of navigating in a three dimensional medical image model, and

FIGS. 5 and 6 show other examples of the implementation of the navigation method.

### DESCRIPTION OF EMBODIMENTS

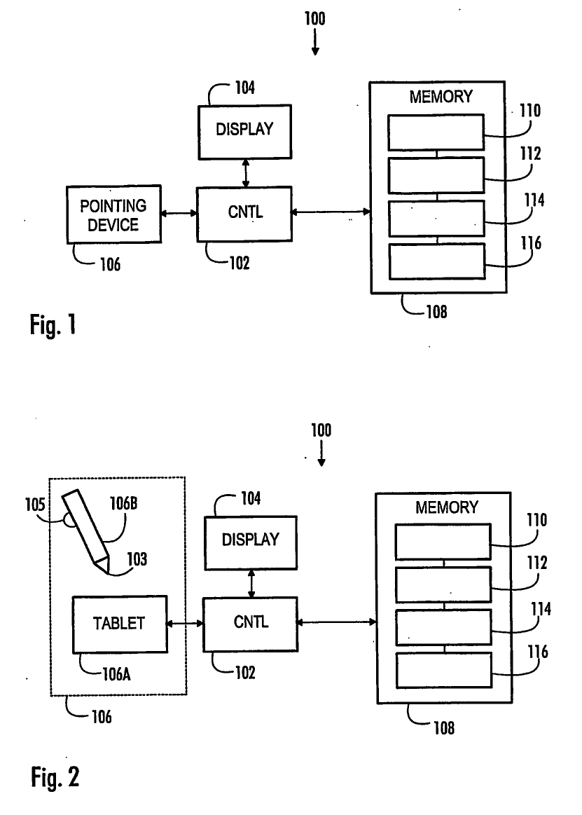

With reference to FIGS. 1 and 2, let us next study the examples of a system in which the embodiments of the invention can be applied. FIGS. 1 and 2 illustrate the structure of the system for navigating in a three-dimensional medical image model. However, the embodiments are not limited to the systems described in these examples; on the contrary, a person skilled in the art is able to apply the inventive solution also to other systems.

The system 100 for navigating in real time in a three-dimensional medical image model shown in FIGS. 1 and 2 comprises a control unit 102, a display 104, a pointing device 106 and a memory 108.

The control unit 102 is connected to the display 104, to the pointing device 106 and to the memory 108. The control unit 102 refers to blocks controlling the operation of the system 100, and is nowadays usually implemented as a processor and software, but also different hardware implementations are feasible, e.g. a circuit built of separate logics components or one or more client-specific integrated circuits (Application-Specific Integrated Circuit, ASIC). A hybrid of these implementations is also feasible. The control unit 102 accesses the memory 108 during executing operations of the system.

Typically, the display 104 is a color display monitor. In one embodiment of the invention, the display 104 is implemented with a contact surface thus forming a touch screen. In the touch screen, the contact surface is on top of the display 104, for example. The control unit 102 displays images on the display 104.

The pointing device 106 comprises means with which the user is able to use the system 100. There can additionally be other user interface parts such as a keyboard or a mouse in the system 100. In the embodiment shown in FIG. 1, the pointing device 106 is for example a pen, a joystick, a stylus or a track ball, which provides input signals to the control unit 102. The input signals are information about the orientation of the pointing device 106, for example. Also, information about a position and orientation, such as a tilt angle, and pressure of the pointing device 106 are provided to the control unit 102. In an embodiment shown in FIG. 2, the pointing device 106 comprises a 106A and a pen 106B. The tablet 106A is, for example, a graphics tablet. Such graphics tablet may be, for example, an Intuos 2 graphics tablet including a tilt sensitive pen that is manufactured by Wacom Co, Ltd. In one embodiment, the tablet 106A may be a contact sensitive graphics tablet and the pen 106B is a wireless pen, for example. The tablet 106A provides in real time position information of the pen 106B tip 103 on the tablet 106A surface to the control unit 102. Also, information about the pen 106B tilt, orientation and pressure is provided to the control unit 102 by means of the tablet 106A and the pen 106B.

The medical image model is stored in the memory 108 of the system, for example. The memory 108 of the system may comprise memory blocks 110-116, in which different data is stored. The memory blocks 110-116 comprise, for example, annotated data from earlier sessions, orientation view data, 3D medical image data sets and representative working results, such as optimal surgical trajectory data. It is possible that the medical image model is created of two-dimensional medical image slices in the system. The two-dimensional medical image slices or three-dimensional medical image models are transferred to the system by means known per se, for example, from another system, such as PACS (Picture Archiving Communications System), or a device. The three-dimensional medical image model is created of two-dimensional MRI medical image slices, for example. It is possible that a number of MRI image slices of the target area of a patient are obtained at given intervals. The MRI image slices are taken so that the entire viewed target area of the patient is covered. As a result, a stack of two-dimensional image slices is taken together thus outlining the entire three-dimensional volume of the target area.

The control unit 102 is configured to display an orientation view of the three-dimensional medical image model on the display 104. The orientation view is, for example, a surface view of the medical image model. Then a location related to the displayed orientation view of the medical image model is adjusted based on the pointing device 106 alignment. The pointing device 106 is, for example, a pen 106B and a tablet 106A, the pointing device 106 alignment thus meaning the pen 106B tip 103 position, the pen 106B orientation or the pen 106B tilt angle on the tablet 106A surface. The location related to the displayed orientation view of the medical image model is a viewpoint or a point from which the user wishes to start navigating the three-dimensional medical image model. With the pointing device 106 alignments, the viewpoint to the model may be rotated thus causing the orientation view rotating at the same time. The user may, for example, move the pen 106B on the tablet 106A surface thus causing the orientation view of the three-dimensional medical image model to rotate horizontally on the display 104. The tilting of the pen 106B in relation to the tablet 106A surface may, for example, cause the orientation view of the three-dimensional medical image model to rotate vertically on the display 104. The speed and amount of the rotation depends on the pen 106B tilt angle, for example.

After the location is adjusted, the control unit 102 is further configured to display an inside view from the location into the three-dimensional medical image model. The inside view of the medical image model comprises one or more medical image slices or other reconstructions as seen from the selected location. The one or more medical image reconstructions are rendered with respect to the orientation view of the three-dimensional medical image model. As a given number of the medical image reconstructions are displayed as the Inside view, an effect of navigating through the three-dimensional medical image model is created. The viewing direction to the inside view of the three-dimensional medical image model is adjusted based on the orientation of the pointing device 106. The tilt angle of the pointing device 106 is between the pen 106B and the tablet 106A surface, for example. The orientation view of the medical image model stays static while the viewing direction to the inside view of the medical image model is adjusted with the pointing device 106.

In an embodiment, it is feasible that the inside view into the three-dimensional medical image model proceeds deeper into the medical image model depending on a pressure against the pointing device 106. The pointing device 106 being a pen 106B and a tablet surface 106A, then the medical image model may proceed deeper into the medical image model depending on the pressure between the pen 106B and the tablet surface 106A. At the beginning of the navigation into the inside view only the first few medical image slices near the surface of the three-dimensional medical image model are displayed, for example. Then, when the pen 106B tip 103 is pressed against the tablet 106A surface or an additional adjustment device 105, such as a joystick or a thumbwheel of the pen 106B is used, for example, the inside view changes such that the image slices deeper in the three-dimensional medical image model are displayed instead. The adjustment device 105 integrated to the pen 106B may be independent of the pen 106B orientation and movements. With the adjustment device 105 different parameters of the three-dimensional medical image model, such as depth, contrast, transparency and/or threshold of the navigated image slices may be adjusted independent of the orientation of the pen 106B. For example, turning the thumbwheel may be used to adjust the viewpoint to the inside view into the desired depth and to remain in that depth regardless of the movements of the pen 106B.

The displayed medical image reconstructions or slices of the inside view are two-dimensional, for example. The orientations of the medical image reconstructions displayed on the display 104 are related to the axis of the pen, for example. The orientations of the medical image reconstructions are selected with the pointing device 106 or by other user interface parts, for example.

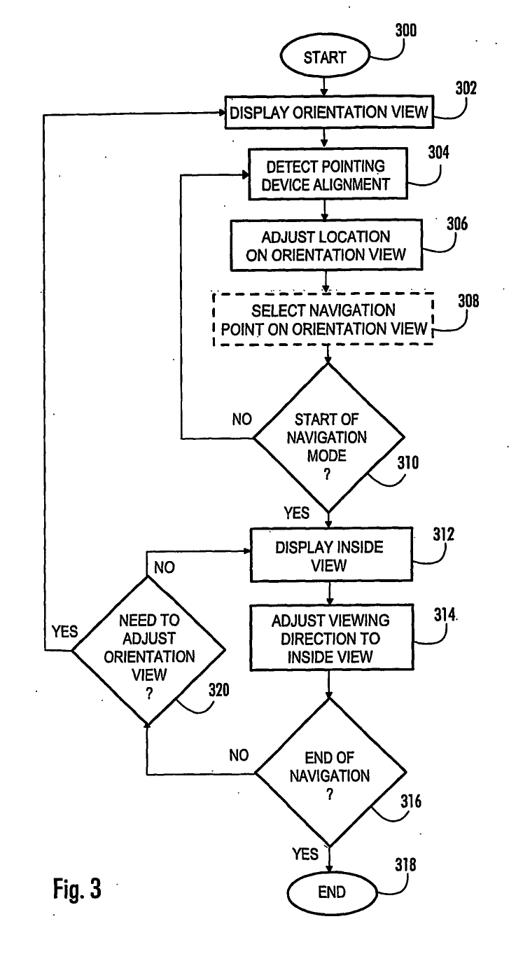

FIG. 3 shows an example of the method for navigating in a three-dimensional medical image model. The dashed lines are illustrating an optional method step. The method starts in 300 wherein the navigation system is ready for use. The desired three-dimensional medical image model is selected and in 302, the orientation view of the three-dimensional medical image model is displayed on the display.

In 304, the control unit detects the pointing device alignment. The control unit detects the pointing device movement and/or orientation. When the pointing device is a pen and a tablet, then the pen tip movement on the tablet surface and the pen tilt angle with reference to the tablet surface is detected.

In 306, the location related to the orientation view of the three-dimensional medical image model is adjusted. The adjustment is carried out with the pointing device. The three-dimensional medical image model is rotated vertically, horizontally and/or laterally by means of the pointing device. When the pointing device is a pen and a tablet, then the tilting of the pen in relation to the tablet would cause the viewpoint to the three-dimensional medical image model to rotate laterally or vertically, for example. The moving of the pen tip on the tablet surface would in turn cause the three-dimensional medical image model to rotate horizontally, for examples.

The reference point selection, in 308, is an alternative method in one embodiment of the invention. The reference point is displayed on the display play on the orientation view of the three-dimensional medical image model. As the pointing device is moved or tilted, the reference point also changes position on the orientation view of the three-dimensional medical image model. The reference point may be displayed with a cursor or the like on the display. When the reference point is at a desired place, at a possible treatment area, for instance, the reference point is selected to act as a navigation point. The control unit based on the pointing device, for example, detects the selection of navigation point.

As the desired orientation of the three-dimensional medical image model is adjusted then, in 310, the control unit detects a start of a navigation mode. The start of the navigation mode is detected based on an input, for example, from the pointing device. If the start of the navigation mode is not detected then 304 and 306 may be executed. When the start of the navigation mode is detected based on depressing a button of the pointing device, for instance, then 312 is entered. In 312, the inside view of the three-dimensional medical image model is displayed on the display. The inside view of the three-dimensional medical image model comprises one or more medical image reconstructions, for example, and the inside view into the medical image model is displayed related to the location of a navigation point. The number of medical image reconstructions displayed on the display can be predetermined in the settings of the navigation system, for example. It is also feasible that the number of the medical image reconstructions displayed on the display is altered during the navigation itself.

In 314, the viewing direction to the inside view of the three-dimensional medical image model is adjusted based on a detected orientation of the pointing device. The orientation of the pointing device may be based on the detected tilt angle and direction between the pen and the tablet surface, for example. The tilting of the pointing device causes the orientation of the one or more displayed medical image slices to change, for example. The tilt angle is determined based on the pointing device orientation, for example. The pointing device being a pen and a tablet, then the tilt angle would be between the pen and the tablet surface, for instance. It is also feasible that more medical image reconstructions deeper in the three-dimensional medical image model are displayed when pressure against the pointing device is detected For instance, the user of the navigation system can press a pen tip against a tablet surface and thus cause other medical image reconstructions in different depths than the previously displayed medical image slices to appear on the display.

In an embodiment, it is possible that the medical image model comprises one or more medical image slices and the adjusting of the viewing direction to the inside view of the medical image model comprises rendering of the medical image slices with respect to the location related to the displayed orientation view of the medical image model. The medical image slices may be generated from two-dimensional image data, for example. The rendered medical image slices are oriented in relation to the detected orientation of the pointing device, for example. In an embodiment of the invention, the rendered medical image slices are orthogonal planes, for example three planes, one of the planes being perpendicular with the axis oriented in relation to the detected orientation of the pointing device.

In 316, if the control unit detects the end of the navigation, then 318 is entered wherein the navigation ends. Otherwise, 310 and 312 may be repeated. It is also possible, that while navigating the user wishes to adjust the location related to the displayed orientation view again. This is possible in a situation where the user, such as a neurosurgeon, discovers, during the navigation mode, that the location related to the displayed orientation view of the medical image model selected in 306, should be changed. If the medical image model illustrates a patients brain having a tumour, for example, then the surgeon may wish to search for an optimum approach to the tumour in order to plan a surgical operation, for example. Then, from 320 it is moved to 302, 304 and 306, in which the orientation view orientation is adjusted, and then back to 312 and 314, in which the actual navigation is performed.

The user may adjust the location related to the displayed orientation view several times during the navigation In order to find an optimum view for the navigation. For example, a brain of a patient comprises many important neurological centres that have to be avoided. Also, the intracranial space around and within the brain is full of fluid spaces that are filled with clear cerebrospinal fluid. It is possible to move within these spaces from point to point using many surgical techniques including microsurgery and endoscopy. The method, as described above, offers a simple way to intuitively plan these complex surgical operations.

It is possible, that the user adjusts the displayed orientation view by giving signals with an input device and uses another input device, such as the pen pointing device to navigate. Thus, several different input devices may be used for controlling the navigation method. The user may adjust the displayed orientation view at any time, even at the same time during the navigation mode.

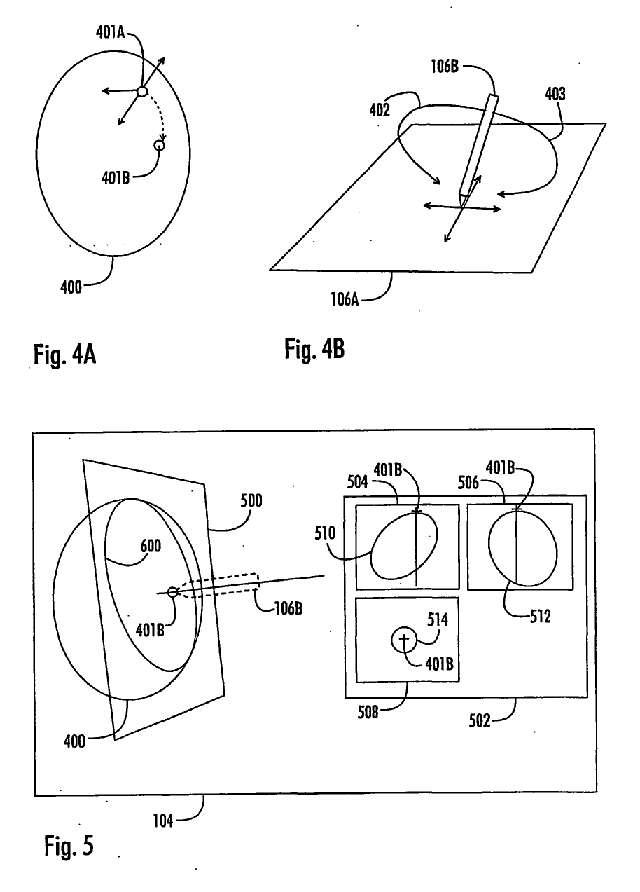

FIGS. 4A and 4B show examples of the implementation of the method of navigating in a three-dimensional medical image model. The FIG. 4A shows an example of how to select the reference point 401A and the navigation point 401B on the orientation view 400 of the three-dimensional medical image model shown on the display. In FIG. 4B, it is illustrated how the pointing device 106B may be moved along the surface of the tablet 106A. The arrows 402 and 403 are illustrating how the pointing device 106B may be tilted in relation to the tablet 106A. The pointing device 106B may be moved along the surface of the tablet 106A in any desired directions, for example.

As the pointing device 106B is aligned on the tablet surface, by moving or tilting, for example, the reference point 401A on the orientation view of the three-dimensional image model moves accordingly. When the desired point is found, this point is locked as a navigation point 401B. In FIG. 4A, the dashed arrow is illustrating the route that the place of the reference point 401A has been travelling before the locking of the navigation point 401B. Then, as the pointing device 106B is aligned, by tilting for example, an inside view based on the alignment of the pointing device 106B is shown on the display.

FIG. 5 illustrates examples of inside views shown on the display 104. The pointing device 106B is shown in dashed lines because typically it is not shown in the inside view of the medical image model. However, it is also possible that a symbol representing the pointing device is shown on the display 104 as well. In FIG. 5, the selected navigation point 401B is shown on the orientation view 400 of the medical Image model. A circle like pattern illustrates the medical image slice 600 that is shown on the display 104. A frame 500 around the medical image slice 600 is for clarifying the orientation of the medical image slice 600 with reference to the orientation view 400 of the medical image model.

Another inside view window 502 is shown in FIG. 5, wherein frames 504, 506, 508 and medical image slices 510, 512, 514 from different points of view are shown. The inside view window 502 consists of orthogonal slices 510, 512, 514 along and across the axis of the pointing device 106B. In the medical image slices 510, 512, 514 there is also shown the place of the navigation point 401B in each of the slices 510, 512, 514. The lines departing from the navigation point 401B in frames 504 and 506 are thus continuations of the axis of the pointing device inside the 3D medical image model. The medical image slices 510 and 512 in frames 504 and 506 are orthogonal slices along the axis of the pointing device. In this example, the medical image slice 514 in frame 508 is a slice perpendicular to the axis of the pointing device 106B and the depth of the medical image slice 514 may be adjusted, for example, by using the thumbwheel as described above. In the exemplary embodiment of FIG. 5, the medical image slice 514 is the same medical image slice 600 that is shown on the left side of the display 104.

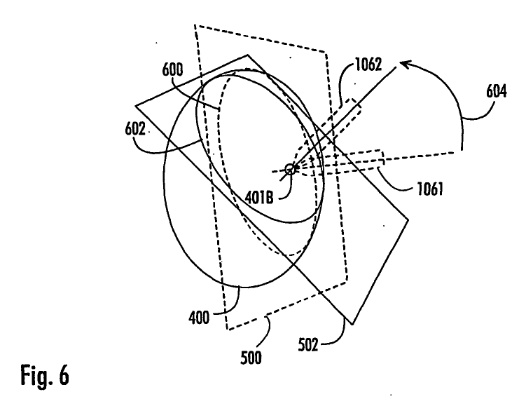

In FIG. 6, there is illustrated how the movement of the pointing device may cause the orientation of the medical image slice to change on the display with respect to the orientation view 400 of the medical image model. At first, the pointing device's position is as illustrated by the dashed lines numbered with 1061. The medical image slice 600 and the frame 500 marked with dashed lines are illustrating the orientation of the slice 600 and the frame 500 when the pointing device's position is at 1061. The arrow 604 shows how the pointing device's position changes from 1061 to 1062. In this example, the pointing device is being tilted upwards. The movement of the pointing device causes the orientations of the medical image slice 600 and the frame 500 to change. The new orientations of the medical image slice 602 and the frame 502 are shown with continuous lines. Thus, the moving of the pointing device causes the medical image slice to change orientation in relation to the orientation view 400 of the medical image model.

In FIGS. 5 and 6, there was shown one medical image slice with reference to the orientation view of the medical image model as an example. However, it is possible that more than one medical image slice or medical image reconstruction set is shown at the same time on the display while navigating the 3D medical image model. The number of medical image slices or reconstructions may be predetermined by the user of the system or changed during the actual navigation. In an embodiment of the invention, also such is possible that some of the medical image slices shown on the display are shown using different filtering or display parameters than the other medical image slices, for example.

In an embodiment, it is also feasible that any data related to the navigated three-dimensional medical image model is recorded to the memory of the system. Such data may comprise one or more images, audio, video, annotation data or any combination thereof. Thus, the data may comprise medical image slices or reconstruction sets at any desired viewpoints and also annotation related to the images. The user, such as a surgeon, may wish to record such data at any time while navigating the three-dimensional medical image model. It is possible to record the whole navigation session including annotations on the important items made by a surgeon while navigating the medical image model. The recording may comprise a video, movement or displaying parameters or any other parameters needed to reconstruct the complete navigation session later. Thus, a user of the system can make comprehensive records of the navigation sessions and use the records at any time later, especially during a later surgical planning session when the patient is present, for example.

The method provides a possibility to neuroradiological conferences that can be used to plan the procedures, such as an approach to a tumour and the work within the tumour. The surgical plan can be made during the navigation, as a natural part of it, with the neurosurgeon consulting the neuroradiologist, for instance. The data that has been recorded during the navigation may be accessed later and be shown in many clinical settings, including neuroconsultation, surgical planning and patient education. Also, different parties, such as experts of certain medical areas, may add any annotations to the recorded data later. The recorded data may be printed out or saved as part of patient medical history files. This makes it possible to easily show to a patient or to an insurance company, for example, any relevant information about the medical procedures concerning an individual patient. For example, any risks and complications that may be involved in individual procedures may be recorded. The recorded data relating to the navigation method may also comprise data about the patient's approvals to take the certain risks and complications involved in certain procedures. The data and the navigation method may also be easily used for educational purposes.

Even though the invention is described above with reference to an example according to the accompanying drawings, it is clear that the invention is not restricted thereto but it can be modified in several ways within the scope of the appended claims.

1-35. (canceled)

36. A method for navigating in real time in a three dimensional medical image model, the method comprising:
displaying an orientation view of the medical image model on a display;
adjusting a location on the displayed orientation view of the medical image model based on detected movement of a pointing device in relation to a worksurface for selecting a navigation point;
rotating the displayed orientation view on the display as the location on the orientation view is adjusted based on the detected movement of the pointing device in relation to the worksurface;
locking the current location on the orientation view as the navigation point based on detected control command from the pointing device;
displaying an inside view related to the navigation point into the medical image model when the navigation point is locked; and
adjusting viewing direction to the inside view of the medical image model based on detected changes in orientation of the pointing device in relation to the worksurface.

37. The method of claim 36, wherein the orientation view is a surface view of the medical image model.

38. The method of claim 36, wherein the pointing device is a pen, a stylus or a pen-like instrument and the worksurface is a tablet surface.

39. The method of claim 38, wherein the detected changes in orientation used in adjusting the viewing direction to the inside view of the medical image model comprise the detected change of a tilt angle and change of orientation between the pen and the tablet surface.

40. The method of claim 38, the method further comprising detecting the movement of the pointing device in relation to the worksurface on the basis of changes of the pen tip position on the tablet surface.

41. The method of claim 38, the method further comprising detecting the movement of the pointing device in relation to the worksurface on the basis of changes of the pen tilt angle in relation to the tablet surface.

42. The method of claim 38, the method further comprising proceeding the inside view of the medical image model deeper into the medical image model depending on the pressure between the pen and the tablet surface.

43. The method of claim 36, wherein the pointing device comprises an adjusting device and the method further comprising adjusting different parameters of the medical image model by the adjustment device.

44. The method of claim 43, the method further comprising adjusting the parameters independently of the orientation of the pointing device by the adjustment device.

45. The method of claim 43, wherein the parameters that are adjusted by the adjustment device are used for proceeding the inside view deeper into the medical image model or for adjusting transparency, contrast and/or threshold of the medical image model.

46. The method of claim 36, wherein the inside view of the medical image model comprises one or more medical image slices or other reconstructions and the adjusting of the viewing direction to the inside view of the medical image model comprises rendering of the medical image slices with respect to the navigation point related to the orientation view of the medical image model.

47. The method of claim 46, the method further comprising generating said one or more medical image slices from two-dimensional image data.

48. The method of claim 46, the method further comprising orienting the rendered medical image slices or other reconstructions in relation to the detected orientation of the pointing device in relation to the worksurface.

49. The method of claim 46, wherein the rendered medical image slices are three orthogonal planes, one of the planes being perpendicular with the axis oriented in relation to the detected orientation of the pointing device.

50. The method of claim 36, wherein adjusting the location on the displayed orientation view of the medical image model comprises synchronously rotating a viewpoint to the orientation view of the medical image model on the display.

51. The method of claim 36, the method further comprising recording data related to the navigated three-dimensional medical image model to a memory.

52. The method of claim 51, wherein the recorded data comprises one or more images, audio, video, annotation data or any combination thereof.

53. A system for navigating in real time in a three-dimensional medical image model, the system comprising a control unit for controlling the functions of the system, a pointing device operated with a worksurface and being connected to the control unit, and a display connected to the control unit, the control unit being configured to:
display an orientation view of the medical image model on the display;
adjust a location on the displayed orientation view of the medical image model based on detected movement of the pointing device in relation to the worksurface for selecting a navigation point;
rotate the displayed orientation view on the display as the location on the orientation view is adjusted based on the detected movement of the pointing device in relation to the worksurface;
lock the current location on the orientation view as the navigation point based on detected control command from the pointing device;
display an inside view related to the navigation point into the medical image model when the navigation point is locked; and
adjust viewing direction to the inside view of the medical image model based on detected changes in orientation of the pointing device in relation to the worksurface.

54. The system of claim 53, wherein the orientation view is a surface view of the medical image model.

55. The system of claim 54, wherein the pointing device comprises a pen, a stylus or a pen-like instrument and the worksurface is a tablet surface.

56. The system of claim 55, wherein the control unit is configured to adjust the viewing direction to the inside view of the medical image model based on a detected changes in orientation between the pen and the tablet surface, the orientation being a tilt angle and direction between the pen and the tablet surface.

57. The system of claim 55, wherein the control unit is configured to detect the movement of the pointing device in relation to the worksurface on the basis of changes of the pen tip position on the tablet surface.

58. The system of claim 55, wherein the control unit is configured to detect the movement of the pointing device in relation to the worksurface on the basis of changes of the pen tilt angle in relation to the tablet surface.

59. The system of claim 55, wherein the control unit is configured to proceed the inside view of the medical image model deeper into the medical image model depending on the pressure between the pen and the tablet surface.

60. The system of claim 53, wherein the pointing device comprises an adjusting device and the control unit is configured to adjust different parameters of the medical image model by the adjustment device.

61. The system of claim 60, wherein the control unit is configured to adjust the parameters independently of the orientation of the pointing device by the adjustment device.

62. The system of claim 60, wherein the parameters that are adjusted by the adjustment device are used for proceeding the inside view deeper into the medical image model or for adjusting transparency, contrast and/or threshold of the medical image model.

63. The system of claim 53, wherein the inside view of the medical image model comprises one or more medical image slices or other reconstructions and the control unit is configured to adjust the viewing direction to the inside view of the medical image model by rendering of the medical image slices with respect to the orientation view of the medical image model.

64. The system of claim 63, wherein the control unit is configured to orient the rendered medical image slices or other reconstructions in relation to the detected orientation of the pointing device.

65. The system of claim 63, wherein the rendered medical image slices are three orthogonal planes, one of the planes being perpendicular with the axis oriented in relation to the detected orientation of the pointing device.

66. The system of claim 53, wherein the control unit is configured to adjust the location by rotating a viewpoint to the orientation view of the medical image model on the display.

67. The system of claim 53, the system further comprising a memory and wherein the control unit is configured to record data related to the navigated three-dimensional medical image model to the memory.

68. The system of claim 67, wherein the recorded data comprises one or more images, audio, video, annotation data or any combination thereof.
